(19) United States
(12) Patent Application Publication
(10) Pub. No.: US 2016/0158320 A1
(43) Pub. Date: Jun. 9, 2016

(54) DEVICE AND METHOD FOR THE DELIVERY OF DRUGS FOR THE TREATMENT OF POSTERIOR SEGMENT DISEASE

(71) Applicant: DIRECT CONTACT LLC, SWAMPSCOTT, MA (US)

(72) Inventors: Clyde L. Schultz, Ponte Vedra, FL (US); Jerome J. Schentag, Eggertsville, NY (US)

(73) Assignee: DIRECT CONTACT LLC, SWAMPSCOTT, MA (US)

(21) Appl. No.: 14/961,488

(22) Filed: Dec. 7, 2015

Related U.S. Application Data
(63) Continuation of application No. 12/948,836, filed on Nov. 18, 2010, now abandoned.
Application No. 12/948,836 is a continuation-in-part of application No. 10/821,718, filed on Apr. 9, 2004, now abandoned.
Application No. 10/821,718 is a continuation of application No. 12/202,759, filed on Sep. 2, 2008, now Pat. No. 9,216,106.
Application No. 12/202,759 is a continuation-in-part of application No. 11/102,454, filed on Apr. 9, 2005, now abandoned.
Application No. 11/102,454 is a continuation-in-part of application No. 10/971,997, filed on Oct. 22, 2004, now abandoned.
Application No. 10/971,997 is a continuation-in-part of application No. 10/821,718, filed on Apr. 9, 2004, now abandoned.
(60) Provisional application No. 60/461,354, filed on Apr. 9, 2003.
(60) Provisional application No. 60/461,354, filed on Apr. 9, 2003.

(51) Int. Cl. A61K 38/18; G02C 7/04; C07K 16/22; A61K 9/00; A61K 31/573; A61K 31/713

(52) U.S. Cl. CPC A61K 38/1866; A61K 31/573; A61K 31/713; C07K 16/22; A61K 9/0048; G02C 7/04; C07K 2317/76

(57) ABSTRACT

Hydrogel lenses are infused with a drug for the treatment of posterior segment disease. The lenses are placed in contact with the subject's cornea. Drugs can be passively released from the hydrogel and can migrate around the globe of the eye to the posterior segment.

### CROSS-REFERENCE TO RELATED APPLICATIONS

This application is a continuation of U.S. application Ser. No. 12/948,836, filed Nov. 18, 2010, which is a continuation-in-part of U.S. application Ser. No. 10/821,718, filed Apr. 9, 2004, and claims benefit of U.S. Provisional Application No. 60/461,354, filed Apr. 9, 2003. This application is a continuation of U.S. application Ser. No. 12/202,759, filed Sep. 2, 2008, which is a continuation-in-part of U.S. application Ser. No. 11/102,454, filed Apr. 9, 2005, which is a continuation-in-part of U.S. application Ser. No. 10/971,997, filed Oct. 22, 2004 which is a continuation-in-part of U.S. application Ser. No. 10/821,718, filed Apr. 9, 2004, which claims benefit of U.S. Provisional Application No. 60/461,354, filed Apr. 9, 2003. Each of these applications is hereby incorporated by reference herein.

### BACKGROUND OF THE INVENTION

In general, the invention relates to the fields of hydrogels, drug delivery systems, the treatment of eye disease and, in particular, posterior segment diseases.

Systemic and topical (e.g., via eye drops) administration of drugs for treatment of diseases of the posterior segment of the eye, such as macular degeneration, are often undesirable. These methods typically require higher total doses of the drug because these routes are inefficient at delivering the drug to the posterior segment. Such high doses increase the cost and may also cause side effects such as local inflammation or adverse systemic reactions. In addition, for most topical treatments, the drug is quickly washed out of the eye, limiting the effective time of treatment.

Thus, sustained-release delivery devices that would continuously administer a drug to the eye for a prolonged period of time are desired for the treatment of posterior segment diseases.

### SUMMARY OF THE INVENTION

The present invention features hydrogel drug delivery systems and methods of producing and using such systems for the treatment of disease in the posterior segment of the eye, e.g., the vitreous, retina (including the macula), choroids, sclera, and optic nerve. The systems are based on a hydrogel into which one or more drugs are passively transferred from a dilute solution, e.g., an aqueous solution. When placed in contact with eye tissue, the drug or drugs passively transfer out of the hydrogel to provide treatment of posterior segment diseases. The drugs can be transported around the globe of the eye to the posterior segment without significant entry into the vitreous or the systemic circulatory system.

Accordingly, in one aspect, the invention features a polymeric hydrogel that contains a drug for the treatment of a posterior segment disease, wherein the drug is capable of being passively released in a therapeutically effective amount to treat the posterior segment disease. Exemplary hydrogel materials include a tetrapolymer of hydroxymethylmethacrylate, ethylene glycol, dimethylmethacrylate, and methacrylic acid. Other examples of hydrogels include etafilcon A, vifilcon A, lidofilcon A, vasurfilcon A, and polymacon B. In addition, variations of these polymers formed by the use of different packing solutions (e.g., phosphate-buffered saline and boric acid) in the manufacturing process are also included. The hydrogel may be ionic or non-ionic. In various embodiments, the drug is capable of being passively released into the ocular environment under ambient or existing conditions. In other embodiments, the hydrogel may be shaped as a contact lens, e.g., one capable of correcting vision. Such a contact lens may be capable of correcting vision in the range of +8.0 to −8.0 diopters or may be plano. The contact lens may also have a base curve between 8.0 and 9.0.

The invention further features a method for making a hydrogel drug delivery system by placing the hydrogel, e.g., a contact lens, in a solution containing one or more drugs as described herein, which is passively transferred to the hydrogel. This method may further include the steps of washing the hydrogel in an isotonic saline solution and partially desiccating the hydrogel prior to placement in the solution. The solution may have, e.g., a pH between 6.9 and 7.4, and a drug concentration of between 0.00001 and 10%. In one embodiment, the hydrogel is placed in the solution of drug for at least 30 minutes.

In another aspect, the invention features a method for treating a posterior segment disease. The method includes placing a hydrogel, as described herein, in contact with an eye, wherein the drug or drugs are passively released from the hydrogel to treat the disease. In various embodiments, the posterior segment disease is in the vitreous, retina (e.g., the macula), choroids, sclera, or optic nerve. The hydrogel may passively release, for example, at least 0.0001, 0.0005, 0.001, 0.005, 0.01, 0.05, 0.1, 0.5, 1, 10, 15, 20, 50, 75, 100, 250, 500, or 1000 μg of a drug, and the hydrogel may be placed in contact with the eye for at least 0.5, 1, 1.5, 2, 2.5, 3, 3.5, 4, 4.5, 5, 7.5, 10, 15, or 24 hours.

Exemplary drugs and posterior segment diseases are described herein.

As used herein, by “ambient conditions” is meant room temperature and pressure.

By “existing conditions” is meant in situ in the eye.

By “treating” is meant medically managing a patient with the intent that a prevention, cure, stabilization, or amelioration of the symptoms will result. This term includes active treatment, that is, treatment directed specifically toward improvement of the disease; palliative treatment, that is, treatment designed for the relief of symptoms rather than the curing of the disease; preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

By “ocular environment” is meant the tissues of and surrounding the eye, including, for example, the sclera, cornea, and other tissues of the ocular cavity and the posterior segment.

The “posterior segment” of the eye includes the retina (including the macula), choroids, sclera, and optic nerve.

Exemplary posterior segment diseases include retinal detachment, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema, intraocular tumors, hereditary retinal degenerations, AIDS-related retinitis, posterior segment uveitis, and systemic diseases with retinal manifestations. For the purposes of this invention, glaucoma is not a posterior segment disease.

All percentages described in the present invention are by weight unless otherwise specified.

Other features and advantages of the invention will be apparent from the following description and the claims.

### BRIEF DESCRIPTION OF DRAWINGS

In the drawing,

FIGS. 1A and 1B are photomicrographs of histological slides of retinal tissue from treated (1A) and untreated (1B) samples.

### DETAILED DESCRIPTION

This disclosure provides a polymeric drug delivery system including a hydrogel containing one or more drugs for the treatment of a posterior segment disease. Allowing passive transference of this drug from a dilute solution into the hydrogel produces the delivery system. The hydrogel, when placed in contact with the eye, delivers the drug. The delivery of the drug can be sustained over an extended period of time, which is of particular utility in the eye, which is periodically flushed with tears. This sustained delivery may accelerate the treatment process while avoiding potential damaging effects of localized delivery of high concentrations of drugs compared to, e.g., intravitreal injection or eye drops. Posterior Segment Diseases

Posterior segment diseases to be treated include, for example, retinal detachment, neovascularization, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema (e.g., diabetic), intraocular tumors, retinal degeneration (e.g., hereditary), vascular retinopathy, inflammatory diseases of the retina, AIDS-related retinitis, uveitis, and systemic diseases with retinal manifestations. Neovascularizations include retinal, choroidal, and vitreal. The retinal neovascularization to be treated can be caused by diabetic retinopathy, vein occlusion, sickle cell retinopathy, retinopathy of prematurity, retinal detachment, ocular ischemia, or trauma. The intravitreal neovascularization to be treated can be caused by diabetic retinopathy, vein occlusion, sickle cell retinopathy, retinopathy of prematurity, retinal detachment, ocular ischemia, or trauma. The choroidal neovascularization to be treated can be caused by retinal or subretinal disorders of age-related macular degeneration, diabetic macular edema, presumed ocular histoplasmosis syndrome, myopic degeneration, angioid streaks, or ocular trauma. Other posterior segment diseases are known in the art.

Drug Delivery System

Hydrogels. This invention may employ different polymer compositions. For example, conventional soft contact lenses can be used and can be either ionic or non-ionic hydrogels containing between 10% and 90%, e.g., 24% or 37.5% to 65% or 75%, water by weight and can have any base curve, e.g., from 8.0 to 9.0. The contact lenses may also have the ability to correct vision, for example, over a range of diopters of +8.0 to −8.0. Exemplary hydrogel contact lens materials include etafilcon A, vifilcon A, lidofilcon A, polymacon B, vasurfilcon A, and a tetrapolymer of hydroxymethylmethacrylate, ethylene glycol, dimethylmethacrylate, and methacrylic acid. These materials may also be employed, in other physical forms. Other suitable hydrogel materials are known to those skilled in the art. The hydrogels may be insoluble or may dissolve over time in vivo, e.g., over one day or one week. The drug is passively delivered, for example, by diffusion out of the hydrogel, by desorption from the hydrogel, or by release as the hydrogel dissolves.

The drug delivery system may be produced from a partially desiccated hydrogel (or equivalently a partially hydrated hydrogel). The desiccation step removes, for example, approximately 5%, 10%, 15%, 20%, 25%, 30%, 40%, 50%, 60%, or 75% of the water in the hydrogel. Desiccation can occur, for example, by exposure of the hydrogel to ambient or humidity controlled air, by heating the hydrogel for a specific period of time, or by blowing dried gas, such as N2, over the hydrogel. In one embodiment, the hydrogel is saturated with physiological (isotonic) saline prior to desiccation. The partially desiccated hydrogel is then soaked, e.g., for at least 30 minutes, in a dilute solution of drug, e.g., at a pH between 6.9 to 7.4. In certain embodiments, the drug is transferred to a contact lens from a non-aqueous solvent, e.g., dimethyl sulfoxide, which may be at least partially removed and replaced with an aqueous solution prior to use in a patient. The hydrogels may also be soaked for at least 1 hour, 6 hours, 12 hours, or 24 hours. The concentration of drug into which the hydrogel is placed is typically 0.000001, 0.000005, 0.00001, 0.00005, 0.0001, 0.0005, 0.001, 0.005, 0.01, 0.05, 0.1, 0.5, 1, 5, 10, 15, 20, 50, 75, 100, 250, 500, or 1000 μg/mL. Higher concentrations may also be used, for example, to reduce the soaking time. The drug is passively transferred into the hydrogel. This transfer may occur at least in part by rehydrating the hydrogel. Diffusion of the drug into the water or polymer in the hydrogel may also occur. In alternative embodiments, a fully hydrated or fully desiccated hydrogel is placed in the soaking solution to produce the medicated hydrogel.

Desirably, the concentration of drug transferred to the hydrogel is substantially lower than the solution in which the hydrogel is soaked. For example, the concentration of growth factor in the hydrogel is at least 2×, 5×, or 10× less than that of the soaking solution. Some drugs, however, may have a higher affinity for a hydrogel than the soaking solution, and such a hydrogel will have a higher concentration of drug than the solution in which it was soaked, e.g., at least 2×, 5×, or 10× more. The water content and type of hydrogel, time and conditions, e.g., temperature of soaking, composition of the soaking solution (e.g., ionic strength and pH), and type of drug employed also may influence the concentration of drug in the drug delivery system. Since the water content of the hydrogel may also help to determine the total amount of drug present in a hydrogel, it represents a variable by which to control the amount of drug delivered to a tissue. The production of a hydrogel containing a specified amount of drug can be accomplished by routine experimentation by one skilled in the art. Drugs for the Treatment of Posterior Segment Diseases.

A variety of drugs and drug precursors may be delivered to the posterior segment. In some embodiments, corticosteroids can be delivered via a hydrogel. Corticosteroids (or corticoids) are any steroids (lipids that contain a hydrogenated cyclopentoperhydrophenanthrene ring system) elaborated by the adrenal cortex (except sex hormones of adrenal origin) in response to the release of adrenocorticotrophin or adrenocorticotropic hormone by the pituitary gland, or to any synthetic equivalent, or to angiotensin II. Corticosteroids include but may not be limited to alclometasone dipropionate, amcinonide, amcinafel, amcinafide, beclomethasone, betamethasone, betamethasone dipropionate, betamethasone valerate, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone diacetate, dichlorisone, esters of betamethasone, flucetonide, flucloronide, fluorocortisone, flumethasone, flunisolide, fluocinonide, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, medrysone, meprednisone, methylprednisone, methylprednisolone, mometasone furoate, paramethasone, prednisone, prednisolone, prednisone, triamcinolone acetonide, and triamcinolone.

In one set of embodiments, short term action corticosteroids can be passively transferred from a hydrogel lens around the globe to the posterior segment. Short term action corticosteroids, as used herein, means agents that are metabolized in situ within 2-4 hours. These drugs include beclomethasone, prednisolone, prednisone, fluticasone, budesonide, betamethasone dipropionate, amelometasone, mometasone and ciclesonide. Derivatives of these drugs may also be used. Derivatives include active salts or acids and/or precursors that can metabolize into active compounds.

In other embodiments, classes of drugs include anti-infectives (e.g., antibiotics, antibacterial agents, antiviral agents, and antifungal agents); analgesics; anesthetics; antiallergenic agents; mast cell stabilizers; steroidal and non-steroidal anti-inflammatory agents; decongestants; antioxidants; nutritional supplements; angiogenesis inhibitors; antimetabolites; fibrinolytics; neuroprotective drugs; angiostatic steroids; mydriatics; cyclopegic mydriatics; miotics; vasoconstrictors; vasodilators; anticlotting agents; anticancer agents; antisense agents, immunomodulatory agents; carbonic anhydrase inhibitors; integrin antagonists; cyclooxgenase inhibitors; differentiation modulator agents; sympathomimetic agents; VEGF antagonists; immunosuppresant agents; and combinations and prodrugs thereof. Other suitable drugs are known in the art.

Exemplary drugs include 17-ethynylestradiol, 2-ethoxy-6-oxime-estradiol, 2-hydroxyestrone, 2-propenyl-estradiol, 2-propynl-estradiol, 4,9(11)-pregnadien-17α,21-diol-3,20-dione, 4,9(11)-pregnadien-17a,21-diol-3,20-dione-21-acetate, 4-methoxyestradiol, 5-fluorouracil, 6-mannosephosphate, acetazolamide, acetohexamide, acetylcholinesterase inhibitors, acyclovir, adrenal corticalsteroids, adriamycin, aldesleukin, aldose reductase inhbitors, alkylating agents including cyclophosphamide, alpha-tocopherol, am ifostine, amphotericin B, anastrozole, anecortave acetate, angiostatic steroids, angiostatin, antazoline, anthracycline antibiotics, antibody to cytokines, anticlotting activase, anti-cytomegalovirus agents, antifibrinogen, antineogenesis proteins, arsenic trioxide, asparaginase, atenolol, atropine sulfate, azacytidine, azathioprine, AZT, bacitracin, bacitracin, betamethasone, betaxolol, bexarotene, bleomycin, busulfan, calcium channel antagonists (e.g., imodipine and diltiazem), capecitabine, carbachol, carmustine, cephalosporin antibiotics, chlorambucil, chloramphenicol, chlorpheniramine, chlorpropamide, chlortetracycline, colchicine, cyclooxgenase II inhibitors, cyclopentolate, cyclophosphamide, cyclosporine, cyclosporine A, cytarabine, cytochalasin B, cytokines, dacarbazine, dactinomycin, daunorubicin, demecarium bromide, dexamethasone, diamox, dichlorphenamide, didanosine, dihydroxylipoic acid, diisopropylfluorophosphate, docetaxel, echinocandin-like lipopeptide antibiotics, echothiophateiodide, eliprodil, endostatin, epinephrine, epirubicin hydrochloride, erythromycin, erythropoietin, eserine salicylate, estradiol, estramustine, etanercept, ethisterone, etoposide, etoposide phosphate, etretinate, eucatropine, exemestrane, famvir, fibrinolysin, filgrastim, floxuridine, fluconazole, fludarabine, fluocinolone, fluoromethalone, fluoroquinolone, fluoxymesterone, flutamide, foscamet, fumagillin analogs, fusidic acid, ganciclovir, gemcitabine HCL, gemtuzumab ozogamicin, gentamicin, glipizide, glutathione, glyburide, goserelin, gramicidin, heat shock proteins, heparin, herbimycon A, homatropine, humanized anti-IL-2receptor mAb (Daclizumab), hydrocortisone, hydroxyamphetamine, hydroxyurea, idoxuridine, ifosfamide, imidazole-based antifungals, insulin, interferon alfa-2a, interferon-gamma, interferons, interleukin-2, irinotecan HCL, ketoconazole, leflunomide, letrozole, leuprolide, levamisole, lidocaine, lipid formulations of antifungals, liposomalamphotericin B, lomustine, macrolide immunosuppressants, matrix metalloproteinase inhibitors, medroxyprogesterone, medrysone, melphalan, memantine, mercaptopurine, mestranol, metals (e.g., cobalt and copper), methapyriline, methazolamide, methotrexate, methylprednisolone, minocycline, mitomycin, mitotane, mitoxantrone hydrochloride, mono and polyclonal antibodies, muramyl dipeptide, mycophenolate mofetil, naphazoline, neomycin, nepafenac, neuroimmunophilin ligands, neurotrophic receptors(Aktkinase), neurotropins, nicotinamide (vitamin B3), nimodipine, nitrofurazone, nitrogen mustard, nitrosoureas, norethynodrel, NOS inhibitors, ondansetron, oprelvekin, oraptamers, oxytetracycline, paclitaxel, pentostatin, pheniramine, phenylephrine, phospholineiodine, pilocarpine, pipobroman, platelet factor 4, platinum coordination complexes (such as cisplatin and carboplatin), plicamycin, polymyxin, prednisolone, prednisone, procarbazine, tacrolimus, prophenpyridamine, prostaglandins, protamine, protease and integrase inhibitors, pyrilamine, rapamycin, ribavirin, rimexolone, rituximab, sargramostim, scopolamine, sodium propionate, streptozocin, succinic acid, sulfacetamide, sulfamethizole, sulfonamides, sulfoxazole, superoxide dismutase, suramine, tamoxifen, temozolomide, teniposide, tetracycline, tetrahydrazoline, thalidomide, thioguanine, thymopentin, thyroid hormones, tolazamide, tolbutamide, topotean hydrochloride, toremifene citrate, transforming factor beta2, trastuzumab, triamcinolone, triazole antifungals, trifluorothymidine, triptorelinpamoate, trisodium phosphonoformate, tropicamide, tumor necrosis factor, uracil mustard, valrubicin, VEGF antagonists (e.g., VEGF antibodies and VEGF antisense), vidarabine, vinblastine, vincristine, vindesine, vitamin B12 analogues, and voriconazole, progranulin, taporfin sodium, MIRA-1 (Occulogix), Sirna-027 (Sirna Therapeutics Inc.), F200 (Protein Design Labs Inc), Cand5 (Acuity Pharmaceuticals), H8 (Cancervax Corporation), RetinoStat (Oxford Biomedica PLC), Angiotensin II Inhibitor (Genomed, Inc.), AK-1003 (Akorn, Inc.), NX 1838 (Gilead Sciences Inc.), DL-8234 (Daiichi Pharmaceutical Co. Ltd), Envision TD (Control Delivery Systems, Inc.) and AMD Fab (Hoffmann-LaRoche).

In one embodiment, the drug is an anti-angiogenesis compound, e.g., for treatment of macular degeneration. Anti-angiogenesis compounds may exert their effects by any mechanism, including metalloproteinase inhibitors, monoclonal antibodies (e.g., anti-integrin or anti-VEGF antibodies), calcium channel inhibitors, vascular targeting agents, tetracycline derivatives, PKC inhibitors, IP-10 upregulators, growth factor antagonists, PDGF antagonists, VEGF antagonists, cytotoxics, antiproliferatives, and Na or Ca channel blockers. Exemplary anti-angiogenesis compounds include 2-methoxyestradiol (PANZEM) (EntreMed), A6, ABT-510, ABX-IL8 (Abgenix), actimid, Ad5FGF-4 (Collateral Therapeutics), AG3340 (Agouron Pharmaceuticals Inc. LaJolla, Calif.), alpha5beta1 integrin antibody, AMG001 (AnGes/Daichi Pharmaceuticals), anecortave acetate (Retaane, Alcon), angiocol, angiogenix (Endovasc Ltd), angiostatin (EntreMed), angiozyme, antiangiogenic antithrombin 3 (Genzyme Molecular Oncology), anti-VEGF (Genentech), anti-VEGF Mab, aplidine, aptosyn, ATN-161, avastin (bevacizumab), AVE8062A, Bay 12-9566 (Bayer Corp. West Haven, Conn.), benefin, BioBypass CAD (VEGF-121) (GenVec), MS275291, CAI (carboxy-amido imidazole), carboxymidotriazole, CC 4047 (Celgene), CC 5013 (Celgene), CC7085, CDC 801 (Celgene), Celebrex (Celecoxib), CEP-7055, CGP-41251/PKC412, cilengitide, CM101 (Carbomed Brentwood, TN), col-3 (CollaGenex Pharmaceuticals Inc. Newton, Pa.), combretastatin, combretastatin A4P (Oxigene/Bristol-Myers Squibb), CP-547, 632, CP-564, 959, Del-1 (VLTS-589) (Valentis), dexrazoxane, didemnin B, DMXAA, EMD 121974, endostatin (EntreMed), FGF (AGENT 3) (Berlex (Krannert Institute of Cardiology)), flavopiridol, GBC-100, genistein concentrated polysaccharide, green tea extract, HIF-1 alpha (Genzyme), human chorio-gonadotrophin, IM862 (Cytran), INGN 201, interferon alpha-2a, interleukin-12, iressa, ISV-120 (Batimastat), LY317615, LY-333531 (Eli Lilly and Company), Mab huJ591-DOTA-90 Yttrium (90Y), marimastat (British Biotech Inc. Annapolis, Md.), Medi-522, metaret (suramin), neoretna, neovastat (AEterna Laboratories), NM-3, NPe6, NV1 FGF (Gencell/Aventis), octreotide, oltipraz, paclitaxel (e.g., taxol, docetaxel, or paxene), pegaptanib sodium (Eyetech), penicillamine, pentosan polysulphate, PI-88, prinomastat (Agouron Pharmaceuticals), PSK, psorvastat, PTK787/ZK222584, ranibizumab (Lucentis, Genentech), razoxane, replistatatin (Platelet factor-4), revimid, RhuMab, Ro317453, squalamine (Magainin Pharmaceuticals, Inc. Plymouth Meeting, Pa.), SU101 (Sugen Inc. Redwood City, Calif.), SU11248, SU5416 (Sugen), SU6668 (Sugen), tamoxifen, tecogalan sodium, temptostatin, tetrathiomol, tetrathiomolybdate, thalidomide (EntreMed Inc., Rockville, Md.), thalomid, TNP-470 (TAP Pharmaceuticals Inc. Deerfield, Wis.), UCN-01, VEGF (Genentech Inc. South San Francisco, Calif.), VEGF trap, Vioxx, vitaxin (Ixsys Inc. San Diego, Calif.), vitaxin-2 (MedImmune), ZD6126, and ZD6474. Additionally anti-angiogensis compounds found in vivo and suitable for use in the compositions and methods described herein include angiostatin (plasminogen fragment), metalloproteinase inhibitors (TIMPs), antiangiogenic antithrombin III (aaATIII), pigment epithelial-derived factor (PEDF), canstatin, placental ribonuclease inhibitor, cartilage-derived inhibitor (CDI), plasminogen activator inhibitor, CD59 complement fragment, platelet factor-4 (PF4), endostatin (collagen XVIII fragment), prolactin 16 kD fragment, fibronectin fragment, proliferin-related protein, gro-beta, retinoids, heparinases, tetrahydrocortisol-S, heparin hexasaccharide fragment, thrombospondin-1, human chorionic gonadotropin (hCG), transforming growth factor-beta, interferon alpha/beta/gamma, tumistatin, interferon inducible protein (IP-10), vasculostatin, interleukin-12 (IL-12), vasostatin (calreticulin fragment), kringle 5 (plasminogen fragment), angioarrestin, and 2-methoxyestradiol. Furthermore compounds that inhibit, block, or antagonize the angiogenic activity of the following species in vivo are useable in the methods and compositions described herein: angiogenin, placental growth factor, angiopoietin-1, platelet-derived endothelial cell growth factor (PD-ECGF), Del-1, platelet-derived growth factor-BB (PDGF-BB), fibroblast growth factors: acidic (aFGF) and basic (bFGF), pleiotrophin (PTN), follistatin, proliferin, granulocyte colony-stimulating factor (G-CSF), transforming growth factor-alpha (TGF-alpha), hepatocyte growth factor (HGF)/scatter factor (SF), transforming growth factor-beta (TGF-beta), interleukin-8 (IL-8), tumor necrosis factor-alpha (TNF-alpha), leptin, vascular endothelial growth factor (VEGF)/vascular permeability factor (VPF), midkine, progranulin, rostaporfin, taporfin sodium, MIRA-1 (Occulogix), Sirna-027 (Sirna Therapeutics Inc.), F200 (Protein Design Labs Inc), Cand5 (Acuity Pharmaceuticals), H8 (Cancervax Corporation), RetinoStat (Oxford Biomedica PLC), Angiotensin II Inhibitor (Genomed, Inc.), AK-1003 (Akorn, Inc.), NX 1838 (Gilead Sciences Inc.), DL-8234 (Daiichi Pharmaceutical Co. Ltd), Envision TD (Control Delivery Systems, Inc.) and AMD Fab (Hoffmann-LaRoche).

Many drugs for the treatment of posterior segment disease may be inhibitors of ocular neovascularization. Inhibition may occur through the blocking or regulating of a number of pathways. These mechanisms may be intercellular or intracellular. For instance, the membrane-bound tyrosine kinase receptors VEGFR-1 and VEGFR-2 can be triggered by VEGF to result in activation of an intracellular tyrosine kinase domain and the resulting vascular endothelial cell proliferation. Drugs for the treatment of posterior segment disease may, for example, sequester and/or neutralize VEGF or block VEGFR-2. These drugs include, for example, VEGF-neutralizing oligonucleotide aptamers such as pegaptanib, humanized anti-VEGF monoclonal antibody fragments, such as ranibizumab, receptor analogs such as sFlt-1, and receptor-immunoglobulin fusion proteins. Other drugs may act as inhibitors of the tyrosine kinase signaling cascade or the degradation of VEGF messenger RNA with interfering RNA's. See van Wijngaarden et al, JAMA, Mar. 23/30 2005, vol. 293, No. 12, pp 1509-1513.

In some embodiments, the drug being delivered can be or can include a nucleic acid. The nucleic acid may be, for example, RNA and/or DNA and may be single or double stranded. The nucleic acid component may include any number of base pairs, for example, from 1 to 100, 1 to 1000, 1 to 10,000, 1 to 100,000 or 1 to 1,000,000 base pairs. The nucleic acids may reduce or retard angiogenesis and may act by binding with or blocking receptor sites responsible for promoting angiogenesis. For example, the compound may be an RNA sequence that is an anti-sense antagonist of VEGF. The compound may react with a specific receptor site on the antagonist.

In other embodiments, the drug can be a compound that disrupts a metabolic pathway, for example, the metabolic pathways responsible for neovascular encroachment on the retina. This may include the disruption of enzymatic pathways in the posterior segment, such as occurs in diabetic retinopathy. The nucleic acid compounds may be intracellular or intercellular. In different embodiments, the anti-sense compound can interact with intracellular or intercellular molecules.

In one set of embodiments, an article can be used to introduce a drug for a posterior segment disease wherein the drug is a Vascular Endothelial Growth Factor (VEGF) ligand or ligand complex. The ligand or ligand complex may include any VEGF ligands and ligand complexes, such as, for example, those disclosed in U.S. Pat. No. 6,051,698, which is hereby incorporated by reference herein. These nucleic acid anti-angiogenesis compounds have been shown to be effective in treating, for example, macular degeneration. These compounds (the MACUGEN compounds) and their derivatives may be delivered directly from an article that is in contact with the eye and typically in contact with the cornea for extended periods of time (e.g., >1hr).

As the compounds can be delivered passively from an article, e.g., a contact lens, and over an extended time, in some embodiments the compounds need not be derivitized and may consist of or consist essentially of nucleic acids. For example, the compounds may be void of fluoro groups such as 2′ fluoro groups, may be void of additional 2′ amino modification and may be void of 2′ O methyl modifications. The compounds may also include or be void of high molecular weight or lipophilic compounds that may, for instance, affect the in vivo stability of the compounds. Compounds may or may not include polyalkylene glycol and/or polyethylene glycol components. As the methods of administration described herein can provide, for example, a consistent concentration of drug directly to the eye over an extended period of time, some embodiments eliminate or reduce the need to alter the in vivo stability of the compounds. As nucleic acids are typically water soluble and soluble in isotonic saline, these compounds may be transferred into an article such as a hydrophilic contact lens by, for example, diffusion, or as a component of an aqueous solution that passes into the lens across an osmotic gradient.

In another embodiment, a protein or peptide, such as an anti-angiogenesis protein or peptide, may be delivered to the posterior segment via an article such as a contact lens. The protein may be an antibody or an antibody fragment. For example, another drug that may be used with the system is LUCENTIS (rhuFab V2), from Genentech, which is believed to be an anti-VEGF antibody fragment.

A drug may be admixed with a pharmaceutically acceptable carrier adapted to provide sustained release of the drug. Exemplary carriers include emulsions, suspensions, polymeric matrices, nanoparticles, microspheres, microcapsules, microparticles, liposomes, lipospheres, hydrogels, salts, and polymers with the drug reversibly bound electrostatically, chemically, or by entrapment. A pharmaceutically acceptable carrier may also include a transscleral diffusion promoting agent, such as dimethylsulfoxide, ethanol, dimethylformamide, propylene glycol, N-methylpyrolidone, oleic acid, isopropyl myristate, polar aprotic solvents, polar protic solvents, steroids, sugars, polymers, small molecules, charged small molecules, lipids, peptides, proteins, and surfactants. In other embodiments, a drug may be essentially free of a carrier such as a nanoparticle.

In some embodiments, the use of preservatives is non-ideal as they may transfer to a hydrogel at a disproportionately high concentration and cause cytotoxicity.

One example of a screening test that may be used to determine if a drug can be delivered by a contact lens, or similar, is to test the drug to determine its solubility in a hydrogel. A candidate drug should exhibit adequate aqueous solubility to be dispersed into a hydrophilic contact lens and to later diffuse or transfer from the contact lens to the ocular fluid. The drug should be soluble at a level that allows loading into the contact lens at a concentration adequate to produce an effect on the subject. For example, if a specific contact lens can hold 100 uL of solution and if a target loading level for the candidate drug is 5 nanograms per lens, then the solubility of the drug should be at least 5 ng/100 uL or 50 ng/mL.

If a candidate drug does not meet these solubility requirements, it may be derivitized to alter its solubility. Alternatively, surfactants and/or other solubility enhancers may be employed to improve the solubility of the drug.

Another technique that can be used to evaluate uptake and release of a drug is to expose an article, such as a lens, to a drug and then to evaluate the amount of uptake and release from the article using HPLC. For example, a lens loaded with a candidate drug can be placed in a solution such as artificial lachrymal fluid under ambient conditions. After a fixed period of time, eg, one hour, a sample of the fluid can be analyzed by HPLC to determine the amount of drug that has leached into the solution. Fresh solution can then be provided and additional samples may be analyzed at later times to develop a curve that indicates the amount of drug released over specific time intervals. From this data, one skilled in the art can determine peak dosing periods, overall dose rates and the expected lifetime of the loaded lens. This information can then be used, for example, to develop a loading target for a lens and a wearing schedule for the subject. Lens type can also be evaluated for use with specific drugs.

Similarly, the amount of uptake by a lens can be evaluated by placing a lens in a drug solution and monitoring, by periodic sampling, the amount of drug remaining in solution. Any reduction in drug concentration in the solution may be presumed to have been absorbed by the lens. This information can be used to determine, for example, concentrations and times that may be used for loading a lens with the drug.

In another embodiment, an article, for example a contact lens, can be used to deliver drugs effective for treating “dry eye” or “dry eye syndrome.” Traditionally, dry eye has been treated with the administration of artificial tears. While this treatment may ease symptoms and improve patient comfort, artificial tears do not treat the cause of the condition itself, that is, the inadequate production of lachrymal fluid by the subject. Recently, several drugs have been shown to be effective at treating dry eye. The procedures described herein provide an ideal method for delivering dry eye drugs as the drugs may be provided directly to the target and at a chosen concentration over a pre-determined period of time. For example, a dry eye drug may be delivered via a hydrophilic contact lens. The lens may be loaded with 1 microgram of a drug and a portion of that drug, for example, >50%, >75% or >90%, may be delivered to the eye over a 24 hour period. After delivery of the drug from the lens, the lens may be replaced with a fresh one or the lens may be reloaded with drug.

In some embodiments it may be notable that the drug is an active therapeutic that is delivered by the lens to a portion of the eye that is not in contact with the lens. In this way the drug acts at a site that is not in direct contact with the lens. This may serve, for example, to increase tear production in the subject rather than to simply replace missing lachrymal fluid. This is in contrast to a lens that is treated with a substance, such as a lubricant, e.g., petrolatum or PEG, that is designed to improve the feel of a contact lens on the eye.

The article, for example a lens, may also be used to deliver two or more drugs simultaneously. For example, a dry eye drug may be co-administered with a drug for a posterior segment condition. In another embodiment, two or more dry eye drugs can be co-administered. In another embodiment, a nucleic acid may be co-administered with a protein or polypeptide.

Administration of a dry eye drug via an article such as a contact lens may also ameliorate the dry eye condition by reducing moisture loss that occurs through evaporation. By forming a barrier between the surface of the eye and the air, the amount of surface area of the eye exposed to the air is reduced, resulting in a reduction in evaporative losses. Thus, the article may both deliver a dry eye drug as well as reduce evaporative moisture loss.

Any dry eye drug that can be loaded into or onto the lens may be delivered using this technique. Some of the therapeutic drugs with which the system may be useful include RESTASIS (cyclosporine ophthalmic emulsion), Diquafosol and salts thereof, such as Diquafosol tetrasodium, Rebamipide, OPC-12759, ELIDEL, pimecrolimus ophthalmic suspension, 15-HETE, hydroxyeicosatetraenoic acid, ECABET Sodium, prostaglandins, nicotinic acetylcholine receptor agonists, and phosphodiesterase inhibitors. Some of these compounds are described in U.S. Pat. Nos. 4,753,945, 6,277,855, 6,566,398, 6,645,978, 6,645,994, 6,659,985, and which are incorporated by reference herein.

Other drugs that help to relieve dry eye and may be useful with the invention include, for example, polyvinyl alcohol, hydroxypropyl methylcellulose, polyethylene glycol 400 castor oil emulsion, carboxymethylcellulose sodium, propylene glycol, hydroxypropyl guar, carboxymethylcelluose sodium, white petrolatum, mineral oil, dextran 70, glycerin, and hypromellose. Some other materials that may aid in the treatment of dry eye are flaxseed and fish oils, omega 3 and omega 6 fatty acids, lutein and primrose oil. A drug may be admixed with a pharmaceutically acceptable carrier adapted to provide sustained release of the drug. Exemplary carriers include emulsions, suspensions, polymeric matrices, microspheres, microcapsules, microparticles, liposomes, lipospheres, hydrogels, salts, and polymers with the drug reversibly bound electrostatically, chemically, or by entrapment. A pharmaceutically acceptable carrier may also include a transscleral diffusion promoting agent, such as dimethylsulfoxide, ethanol, dimethylformamide, propylene glycol, N-methylpyrolidone, oleic acid, isopropyl myristate, polar aprotic solvents, polar protic solvents, steroids, sugars, polymers, small molecules, charged small molecules, lipids, peptides, proteins, and surfactants.

By administering short term drugs via hydrogel, the benefits of the drug may be maximized while reducing or eliminating undesirable side effects. Passive release of a compound from a hydrogel can provide for a consistent dosing concentration at the target (posterior segment or anterior segment) even if the compound is metabolized quickly. Because the compound is provided directly to the posterior segment, effects on the vitreous humor, such as elevated intraocular pressure, can be reduced or eliminated. Specific hydrogels and specific concentrations of drugs in hydrogels can be readily determined by those of skill in the art when provided with details regarding the drug, the subject, and the condition being treated.

The use of preservatives is non-ideal as they may transfer to a hydrogel at a disproportionately high concentration and cause cytotoxicity.

Treatment Approaches

To treat a posterior segment disease, the hydrogels of the invention are contacted with the cornea or ocular fluid of an individual. The hydrogels may be employed in an open or closed eye period. When the system is shaped as a contact lens, the lens may simply be placed in the eye normally in order to deliver the drug. The hydrogel may also be part of a bandage or may be adhered (e.g., by adhesives or sutures) to the eye. If the hydrogel is placed internally in a patient, the hydrogel is advantageously biodegradable. The time period over which a hydrogel lens is worn may depend on the level of treatment desired or the amount of drug in the lens. Hydrogels may be considered to be disposable and may be replaced after a specified period of time, e.g., at least 0.5, 1, 1.5, 2, 2.5, 3, 3.5, 4, 4.5, 5, 7.5, 10, 15, or 24 hours. Alternatively, a hydrogel that has a depleted amount of drug may be recycled by soaking the hydrogel again in a solution of drug.

The methods of treatment described herein are capable of delivering a drug to the ocular environment of a patient for a period of time longer than the dwell time achievable by gels or drops. The convenience and simplicity of this system would in many cases enhance patient compliance with therapy.

In certain embodiments, at least 0.001, 0.005, 0.01, 0.05, 0.1, 0.5, 1, 5, 10, 15, 20, 50, 75, 100, 200, 500, 750, or 1000 μg of the drug is released from the hydrogel. This delivery occurs by passive transfer and allows medications to be delivered to the posterior segment. The use of hydrogels of the invention may also allow patients to be treated using fewer applications than with traditional methods. In addition, the drug may be released from the hydrogel at a more rapid rate than the release of the drug into a fixed volume of fluid because as the eye produces tears, the drug released is flushed away from the site of application causing an increase in the relative rate of diffusion of the drug out of the hydrogel. The replenishing action of fluids such as tears may also effectively increase the rate of diffusion of the drug into the fluid and lead to earlier onset of therapeutic activity.

Hydrogels may also be used to deliver intermediate and long acting corticosteroids. For these compounds, total contact time and frequency of contact time may be less than with short acting compounds. A significant portion of the compound stored in the hydrogel can be delivered to the posterior segment and can be helpful in avoiding undesirable side effects. In some instances, greater than 50%, greater than 75% or greater than 90% of the drug carried by the hydrogel can be delivered to the posterior segment.

In another embodiment, medicaments that include vitamins or growth factors may be delivered via a hydrogel. Such compounds may have activity in the anterior or posterior segment of the eye. This may include fat soluble and/or water soluble vitamins. Fat soluble vitamins include, for example, Vitamin A and Vitamin E. Derivatives of these vitamins include active salts thereof.

In another embodiment, medicaments may be steroids selected from classes of steroids including estrogens, androgens, progestagens, glucocorticoids, mineralocorticoids, phytosterols, ergosterols and derivatives thereof.

In another embodiment, the medicament may be chosen from the group consisting of squalene, lanosterol, cholesterol, pregnenolone, 17-hydrosypregnenolone, DHEA, androstenedione, androstanediol and derivatives thereof.

In another embodiment, the medicament may be selected from the group consisting of prednisone, prednisolone, methylprednisolone, betamethasone, dexamethasone, triamcinolone, hydrocortisone, aldosterone, squalene, lanosterol, cholesterol, pregnenolone, 17-hydroxypregnenolone, DHEA, androstenedione, androstanediol, estradiol, estriol, estrone, testosterone, dihydrotestosterone, androsterone, progesterone, 17-hydroxyprogesterone, progestins, cortisol, prednisone, stigmasterol, brassicasterol, ergosterol, ergocalciferol and derivatives thereof.

In another embodiment, the drug may comprise an anti-inflammatory compound. Anti-inflammatory compounds that may be useful include, for example, cyclosporin, sirolimus, rapamycin, cyclophilin A, B, or D inhibitors and derivatives thereof.

Hydrogels may in the form of a contact lens and may be placed on the cornea in a conventional manner. Hydrogels including a drug or drug precursor may be kept in contact with the eye for short or extended periods. For instance, contact times may be greater than one minute, greater than 1 hour, greater than six hours, greater than 12 hours, greater than 1 day or greater than 7 days. Likewise, contact times may be less than one week, less than one day, less than 12 hours or less than six hours. In some embodiments it may be desirable to administer a compound to the posterior segment for only a portion of the 24 hour day or for several different portions of the day. Unlike vitreal injection and other invasive techniques, hydrogels allow for fine tuning of drug administration times. In some cases, the subject can place and remove the hydrogel at various time intervals without supervision or aid from medical personnel. The same hydrogel lens may be placed in contact with the eye one, two, three or more times. For instance, a lens may be contacted with the eye for a first time period and then removed for a second time period before being replaced for a third time period. In some embodiments, specific times of day may be chosen for contact with the subject's eye. For example, a hydrogel lens may be used at night while the subject sleeps and may be removed during the day.

The concentration of a drug that is to be used in a hydrogel is a function of several parameters including the effective concentration at the posterior segment, the rate of release from the hydrogel and the percentage of the released compound that is delivered to the posterior segment. Rate of release is a function of several factors, including the composition of the hydrogel, the composition of the aqueous component of the hydrogel, the kinetic properties of the drug itself and the environment on the cornea of the subject's eye to be treated. Appropriate quantities to infuse into a hydrogel can be facilitated by knowing what the effective dose at the posterior segment is in combination with the knowledge that a significant portion of the drug will be delivered directly to the posterior segment. Typically, these quantities can be determined by routine experimentation.

In some embodiments, such as with classes of corticosteroids, a single hydrogel contact lens may be loaded with about 1 mg of active compound. For instance, experiments have shown that an effective concentration of beclomethasone can be delivered to the posterior segment by treating with a lens that includes about 700 ng of the compound. For prednisolone, 450 ng per lens has been shown to be effective. In each case, about 95% of the drug exited the lens after 2 hours of contact with the cornea. It is believed that the consistent pore size of the hydrogels provides for a consistent delivery rate of a variety of compounds.

To incorporate a compound into a hydrogel, the compound may be provided as a suspension or solution in, for example, water for injection or saline for injection. The compound of interest can be incorporated into the lens by placing it in the solution for a period of several hours. The lens may be partially desiccated by exposing it to air for a short period prior to immersion into the suspension. Results show that with a 1 mL suspension volume and with suspension concentration in the range of from 1 to 5 mg/L that with gentle agitation about 0.07% of the compound is incorporated into the lens after a 3 hour period. Higher concentrations of the compound in the solution and/or longer immersion times do not appear to increase the amount of compound that is infused into the lens.

In one aspect, a method is provided in which a drug can be delivered via a hydrogel lens directly to the posterior segment without significant passage through the vitreous of the eye or through the subject's circulatory system. This pathway can provide important advantages due to the direct delivery to the afflicted segment without entry into the vitreous or the systemic circulatory system. Drugs used for the treatment of posterior segment disease are often introduced via vitreal injection. These methods of delivery may have deleterious effects on portions of the eye that are not the target for treatment. For instance, drugs such as corticosteroids may cause an increase in intraocular pressure that may require additional treatment or may require a reduction in the administration of the drug. By circumventing the vitreous, direct delivery of a drug to the posterior segment around the eye globe can reduce or eliminate these side effects.

The data below indicate that drugs can be delivered passively from a contact lens to the posterior segment (including the retina, macula and optic nerve) by passing around the globe of the eye without significant entry into the vitreous or the subject's systemic circulatory system. The pathway of delivery is believed to be through one or more of three different routes. The first is through the circulatory system of the eye, which is isolated from the rest of the circulatory system of the subject. The second pathway is through the nerve system of the eye, and the third pathway is through the musculature that surrounds the eye and penetrates to the posterior segment.

A series of experiments were designed to determine the efficacy of delivery to the posterior segment from a hydrogel positioned on the cornea. The experiments also measured the amount of drug found in the vitreous humor and in the blood plasma (indicating systemic involvement).

### EXAMPLE

To illustrate the ability to deliver a drug to the posterior segment using a hydrogel, an experiment was designed and completed using a contact lens to provide a drug to the retina. New Zealand White rabbits were treated with VEGF in each eye, followed by treatment with prednisolone in one eye, leaving the other as a control. VEGF is known to lead to edema in the retina and prednisolone is known to interfere with this mechanism. The contact lens was a high water ionic polymer lens (SOFTLENS 66, Bausch and Lomb, Rochester, N.Y.) having a water content of about 66%. Each lens had a diameter of about 13 mm.

Lens Preparation

Lenses were desiccated according to standard manufacturing procedures. Lenses were soaked at room temperature in a 1 mg/mL aqueous solution of VEGF (Sigma) for a period of 12 hours. Similar lenses were then separately soaked at room temperature in a 1 mg/mL aqueous solution of prednisolone for a period of 12 hours in order to load the lenses with the drug.

Lenses containing VEGF were placed on the cornea of each eye for a 4 hour closed-eye period. After removal of these lenses, a prednisolone loaded lens was then placed in the left eye for a 4 hour closed-eye period. The right eye was not treated with prednisolone.

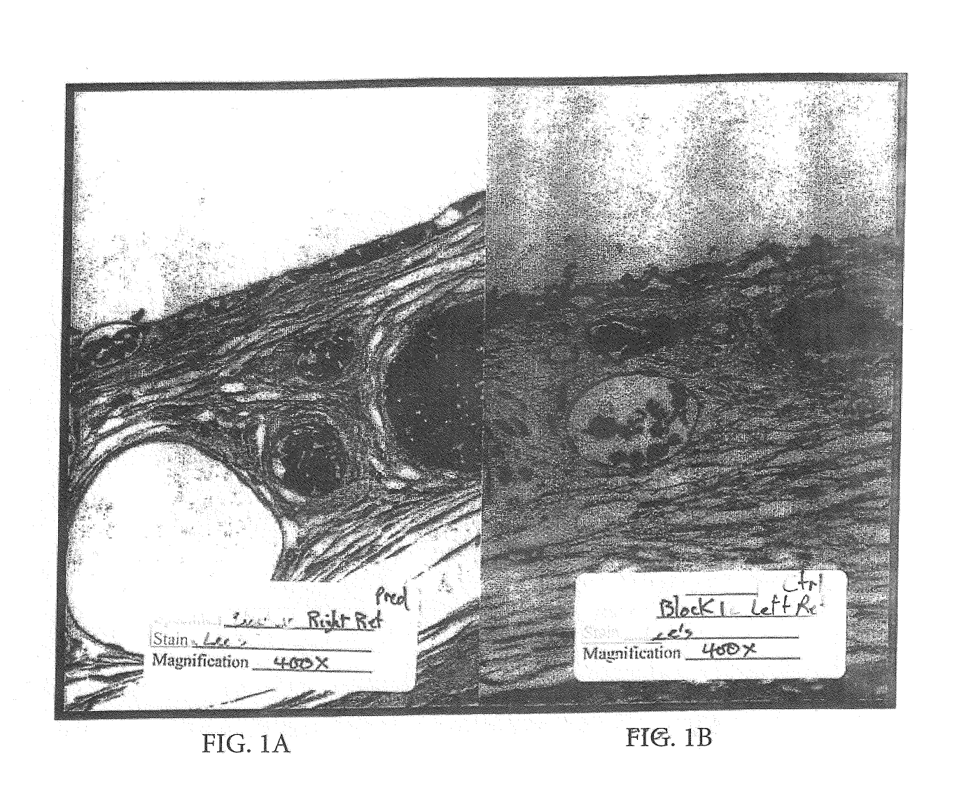

Within 48 hours, the respective retinas from each eye were harvested and cross-sectional slides were prepared using Lee's stain. Photomicrographs (400×) of the respective retinas are provided in FIGS. 1A and 1B. FIG. 1A shows the right retina that received VEGF but no prednisolone. FIG. 1B shows the left retina which received both VEGF and prednisolone. As is evident from the slides, the right retina (FIG. 1A no prednisolone) shows edema as evidenced by the large space that is not apparent in the left retina (FIG. 1B prednisolone). As both eyes were exposed to equal doses of VEGF, the lack of edema in the left retina must be the result of prednisolone being delivered from the lens to the retina.

In the first experiment, rabbits were treated by placing hydrogel lenses on the corneas of the animals for a period of 3 hours. Each lens was infused with about 450 ng of prednisolone incorporated therein. The lenses were placed onto the eyes of anesthetized animals (closed eye period) for the three hour period after which time the lenses were removed. The procedure was repeated about every other day or every three days until 5 applications had been completed.

After completion of the five applications, each eye of the animal was analyzed for prednisolone concentration using LC/MS/MS. The portion of the posterior segment that was analyzed included macula, retina, surrounding muscle, nerve and circulatory, including connective tissues and cells. Sample size typically was about 800 mg. Table 1 provides data for prednisolone concentrations measured in the posterior segment as well as in the aqueous humor. Table 2 provides data for prednisone using the same samples as for Table 1. The limit of quantification for prednisolone was 0.5 ng/mL and for prednisone was 0.05 ng/mL. A data point of “BLOQ” indicates non-detectable levels of the compound were found for that data point.

In a second experiment, a different set of rabbit subjects were treated by placing contact lenses infused with about 700 ng beclomethasone onto each cornea. The procedure was identical to that for prednisolone as described above. Table 3 provides beclomethasone concentrations found in the posterior segment as well as in the vitreous humor. Table 4 provides data for 17-Beclomethasone mono-proprionate, a desirable metabolite of beclomethasone. The data generated for tables 3 and 4 are from the same samples. The limit of quantification for both beclomethasone and 17-beclomethasone mono-proprionate was 0.05 ng/mL. A data point of “BLOQ” indicates non-detectable levels of the compound.

TABLE 1
Prednisolone

| | | Posterior | |
|---|---|---|---|
| | | Segment | Vitreous Humor |
| | | Concentration | Concentration |
| Animal Number | Eye | (ng/G) | (ng/mL) |
| A1 (5) | OS | 74.8 | BLOQ |
| A1 (5) | OD | 26.8 | BLOQ |
| A2 (5) | OS | 166 | BLOQ |
| A2 (5) | OD | 40.8 | BLOQ |
| C2 (5) | OS | 130 | BLOQ |
| C2 (5) | OD | 113 | BLOQ |
| C3 (2) | OS | 31.2 | BLOQ |
| C3 (2) | OD | 26.0 | BLOQ |

TABLE 2
Prednisone

| | | Posterior | |
|---|---|---|---|
| | | Segment | Vitreous Humor |
| | | Concentration | Concentration |
| Animal Number | Eye | (ng/G) | (ng/mL) |
| A1 (5) | OS | BLOQ | 0.219 |
| A1 (5) | OD | BLOQ | BLOQ |
| A2 (5) | OS | BLOQ | BLOQ |
| A2 (5) | OD | BLOQ | BLOQ |
| C2 (5) | OS | BLOQ | 0.147 |
| C2 (5) | OD | BLOQ | BLOQ |
| C3 (2) | OS | BLOQ | BLOQ |
| C3 (2) | OD | BLOQ | BLOQ |

TABLE 3
Beclomethasone

| | | Posterior | |
|---|---|---|---|
| | | Segment | Vitreous Humor |
| | | Concentration | Concentration |
| Animal Number | Eye | (ng/G) | (ng/mL) |
| B1 (5) | OS | BLOQ | BLOQ |
| B1 (5) | OD | 1.55 | BLOQ |
| B2 (5) | OS | 27 | BLOQ |
| B2 (5) | OD | 6.54 | BLOQ |
| C4 (5) | OS | BLOQ | BLOQ |
| C4 (5) | OD | BLOQ | BLOQ |
| B3 (2) | OS | 12.1 | BLOQ |
| B3 (2) | OD | 9.3 | BLOQ |

TABLE 4
17-Beclomethasone mono-proprionate

| | | Posterior | |
|---|---|---|---|
| | | Segment | Vitreous Humor |
| | | Concentration | Concentration |
| Animal Number | Eye | (ng/G) | (ng/mL) |
| B1 (5) | OS | 3.94 | BLOQ |
| B1 (5) | OD | 9.2 | 0.727 |
| B2 (5) | OS | 57.8 | BLOQ |
| B2 (5) | OD | 23.6 | 0.107 |
| C4 (5) | OS | 1.26 | 0.0678 |
| C4 (5) | OD | 1.64 | BLOQ |
| B3 (2) | OS | 29.4 | 0.190 |
| B3 (2) | OD | 80.8 | 0.0584 |

As can be seen from the results provided in Table 1 there was significant delivery of prednisolone to the posterior segment. The average concentration of prednisolone in the posterior segment was greater than 10% of the amount provided in the hydrogel lenses (five treatments). Treatment with beclomethasone resulted in delivery of beclomethasone to the posterior segment and even greater concentrations of 17-beclomethasone mono-proprionate in the posterior segment. This indicates the formation of metabolites from the parent drug in the posterior segment. The presence of metabolites, as well as the parent compound, in the posterior segment indicates that higher doses of the drug may be delivered to obtain efficacious levels of both the parent compound and the metabolite. Lower dosage levels will typically be used for drugs that do not produce desirable metabolites at the posterior segment.

Although not shown in the tables, plasma analysis indicated an absence (none detected) of the four compounds in the circulatory system. Thus both the vitreous humor and the blood plasma contained less than 1% of the drug concentration present in the hydrogel at the start of treatment. Of the total amount of drug delivered to the subject from the hydrogel, more than 10% of the drug was directed to the posterior segment without detectable entry into the vitreous or the systemic circulatory system. This indicates a surprisingly targeted approach using a passive, non-invasive method of drug delivery to the posterior segment. The metabolite results also indicate that a drug precursor may be delivered from a hydrogel and can be converted to an active compound in the posterior segment itself.

In one embodiment, the drug will penetrate the ocular tissue and migrate into the aqueous humor of the eye. Over time, the concentration of the drug will increase such that ocular tissue in the posterior segment of the eye will come into contact with the drug. The drug may have effects on other types of structures, cells, or tissues that may be present at the time of or prior to administration of the drug.

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention. Other embodiments are within the claims.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated in their entirety herein by reference.

What is claimed is:

1. A polymeric hydrogel contact lens comprising a drug for the treatment of a posterior segment disease, wherein when said contact lens is placed on a patient's cornea, and said drug is passively released from the contact lens and contacts the posterior segment of the patient's eye in a therapeutically effective amount to ameliorate and/or stabilize said posterior segment disease.

2. The contact lens of claim 1 wherein the drug comprises an anti-inflammatory compound.

3. The contact lens of claim 1 wherein the drug is selected from the group consisting of beclomethasone, prednisolone, prednisone, fluticasone, budesonide, betamethasone dipropionate, amelometasone, mometasone, ciclesonide, triamcinolone acetonide, fludrocorisone, flumethasone and derivatives thereof.

4. The contact lens of claim 1 wherein the drug is a steroid selected from the group consisting of estrogens, androgens, progestagens, glucocorticoids, mineralocorticoids, phytosterols, ergosterols and derivatives thereof.

5. The contact lens of claim 1 wherein the drug is selected from the group consisting of cyclosporin, sirolimus, rapamycin, cyclophilin A, B, or D inhibitors and derivatives thereof.

6. The contact lens of claim 1 wherein the drug is an anti-angiogenesis compound.

7. The contact lens of claim 7 wherein the drug is selected from the group consisting of 2-methoxyestradiol (PANZEM) (EntreMed), A6, ABT-510, ABX-IL8 (Abgenix), actimid, Ad5FGF-4 (Collateral Therapeutics), AG3340 (Agouron Pharmaceuticals Inc. LaJolla, Calif.), alpha5betal integrin antibody, AMG001 (AnGes/Daichi Pharmaceuticals), anecortave acetate (Retaane, Alcon), angiocol, angiogenix (Endovasc Ltd), angiostatin (EntreMed), angiozyme, antiangiogenic antithrombin 3 (Genzyme Molecular Oncology), anti-VEGF (Genentech), anti-VEGF Mab, aplidine, aptosyn, ATN-161, avastin (bevacizumab), AVE8062A, Bay 12-9566 (Bayer Corp. West Haven, Conn.), benefin, BioBypass CAD (VEGF-121) (GenVec), MS275291, CAI (carboxy-amido imidazole), carboxymidotriazole, CC 4047 (Celgene), CC 5013 (Celgene), CC7085, CDC 801 (Celgene), Celebrex (Celecoxib), CEP-7055, CGP-41251/PKC412, cilengitide, CM101 (Carbomed Brentwood, Tenn.), col-3 (CollaGenex Pharmaceuticals Inc. Newton, Pa.), combretastatin, combretastatin A4P (Oxigene/Bristol-Myers Squibb), CP-547, 632, CP-564, 959, Del-1 (VLTS-589) (Valentis), dexrazoxane, didemnin B, DMXAA, EMD 121974, endostatin (EntreMed), FGF (AGENT 3) (Berlex (Krannert Institute of Cardiology)), flavopiridol, GBC-100, genistein concentrated polysaccharide, green tea extract, HIF-1 alpha (Genzyme), human chorio-gonadotrophin, IM862 (Cytran), INGN 201, interferon alpha-2a, interleukin-12, iressa, ISV-120 (Batimastat), LY317615, LY-333531 (Eli Lilly and Company), Mab huJ591-DOTA-90 Yttrium (90Y), marimastat (British Biotech Inc. Annapolis, Md.), Medi-522, metaret (suramin), neoretna, neovastat (AEterna Laboratories), NM-3, NPe6, NV1 FGF (Gencell/Aventis), octreotide, oltipraz, paclitaxel (e.g., taxol, docetaxel, or paxene), pegaptanib sodium (Eyetech), penicillamine, pentosan polysulphate, PI-88, prinomastat (Agouron Pharmaceuticals), PSK, psorvastat, PTK787/ZK222584, ranibizumab (Lucentis, Genentech), razoxane, replistatatin (Platelet factor-4), revimid, RhuMab, Ro317453, squalamine (Magainin Pharmaceuticals, Inc. Plymouth Meeting, Pa.), SU101 (Sugen Inc. Redwood City, Calif.), SU11248, SU5416 (Sugen), SU6668 (Sugen), tamoxifen, tecogalan sodium, temptostatin, tetrathiomol, tetrathiomolybdate, thalidomide (EntreMed Inc., Rockville, Md.), thalomid, TNP-470 (TAP Pharmaceuticals Inc. Deerfield, Wis.), UCN-01, VEGF (Genentech Inc. South San Francisco, Calif.), VEGF trap, Vioxx, vitaxin (Ixsys Inc. San Diego, Calif.), vitaxin-2 (MedImmune), ZD6126, and ZD6474. Additionally anti-angiogensis compounds found in vivo and suitable for use in the compositions and methods described herein include angiostatin (plasminogen fragment), metalloproteinase inhibitors (TIMPs), antiangiogenic antithrombin III (aaATIII), pigment epithelial-derived factor (PEDF), canstatin, placental ribonuclease inhibitor, cartilage-derived inhibitor (CDI), plasminogen activator inhibitor, CD59 complement fragment, platelet factor-4 (PF4), endostatin (collagen XVIII fragment), prolactin 16 kD fragment, fibronectin fragment, proliferin-related protein, gro-beta, retinoids, heparinases, tetrahydrocortisol-S, heparin hexasaccharide fragment, thrombospondin-1, human chorionic gonadotropin (hCG), transforming growth factor-beta, interferon alpha/beta/gamma, tumistatin, interferon inducible protein (IP-10), vasculostatin, interleukin-12 (IL-12), vasostatin (calreticulin fragment), kringle 5 (plasminogen fragment), angioarrestin, 2-methoxyestradiol, angiogenin, placental growth factor, angiopoietin-1, platelet-derived endothelial cell growth factor (PD-ECGF), Del-1, platelet-derived growth factor-BB (PDGF-BB), fibroblast growth factors: acidic (aFGF) and basic (bFGF), pleiotrophin (PTN), follistatin, proliferin, granulocyte colony-stimulating factor (G-CSF), transforming growth factor-alpha (TGF-alpha), hepatocyte growth factor (HGF) /scatter factor (SF), transforming growth factor-beta (TGF-beta), interleukin-8 (IL-8), tumor necrosis factor-alpha (TNF-alpha), leptin, vascular endothelial growth factor (VEGF)/vascular permeability factor (VPF), midkine, progranulin, rostaporfin, taporfin sodium, MIRA-1 (Occulogix), Sirna-027 (Sirna Therapeutics Inc.), F200 (Protein Design Labs Inc), Cand5 (Acuity Pharmaceuticals), H8 (Cancervax Corporation), RetinoStat (Oxford Biomedica PLC), Angiotensin II Inhibitor (Genomed, Inc.), AK-1003 (Akorn, Inc.), NX 1838 (Gilead Sciences Inc.), DL-8234 (Daiichi Pharmaceutical Co. Ltd), Envision TD (Control Delivery Systems, Inc.) and AMD Fab (Hoffmann-LaRoche).

8. The contact lens of claim 1 wherein the drug comprises a Vascular Endothelial Growth Factor ligand or ligand complex.

9. The contact lens of claim 1 wherein the drug comprises a nucleic acid.

10. The contact lens of claim 1 wherein the drug comprises an antibody or antibody fragment.

11. The contact lens of claim 1 wherein the drug is a compound that is metabolized in situ in less than 4 hours.

12. The contact lens of claim 1 comrpising a terapolymer of hydroxymethylmethacrylate, ethylene glycol, dimethylmethacrylate and methacrylic acid.

13. The contact lens of claim 1 having a base curve between 8.0 and 9.0.

14. The contact lens of claim 1 wherein the posterior segment disease is selected from retinal detachment, diabetic retinopathy, macular degeneration (e.g., age-related), proliferative vitreoretinopathy, endophthalmitis, retinopathy of prematurity, posterior segment trauma, intraocular lens-related posterior segment complications, retinal vascular diseases, macular edema, intraocular tumors, hereditary retinal degenerations, AIDS-related retinitis, posterior segment uveitis, and systemic diseases with retinal manifestations.

15. The contact lens of claim 1 comprising between 10% and 90% water by weight.
